(19) United States
(12) Patent Application Publication
(10) Pub. No.: US 2008/0268013 A1
(43) Pub. Date: Oct. 30, 2008

(54) Polyethylene Oxide Polymers Including Anti-Inflammatory Glycodendrons

(75) Inventors: Elliot Lorne Chaikof, Atlanta, GA (US); Shyam Mohan Rele, San Dimas, CA (US); Wanxing Cui, Norcross, GA (US); Yves Gnanou, Pessac Cedex (FR); Jeffrey D. Esko, San Diego, CA (US)

Correspondence Address: GREENLEE WINNER AND SULLIVAN P C, 4875 PEARL EAST CIRCLE, SUITE 200, BOULDER, CO, 80301, US

(73) Assignees: EMORY UNIVERSITY, ATLANTA, GA (US); UNIVERSITY OF CALIFORNIA, SAN DIEGO, LA JOLLA, CA (US)

(21) Appl. No.: 11/815,993

(22) PCT Filed: Feb. 10, 2006
(86) PCT No.: PCT/US2006/004707

§ 371 Date: Apr. 14, 2008

Related U.S. Application Data
(60) Provisional application No. 60/651,891, filed on Feb. 10, 2005.

(51) Int. Cl. C08F 251/00; A61F 2/00; C12N 5/06; A61P 37/00; A61P 29/00; A61K 31/765; A61F 2/82

(52) U.S. Cl. 424/423; 527/300; 424/78.37; 435/375

(57) ABSTRACT

Poly(ethylene oxide) (PEO) glycodendrimers can serve as multivalent inhibitors of selectin-mediated leukocyte recruitment regulating inflammatory response. Disclosed are compounds and methods relating to functionalized branched glycopolymers. In embodiments, the compounds are capable of modifying cell adhesion events and inflammatory conditions. In a particular embodiment, a multi-arm PEO polymer with sulfated lactose end groups can specifically inhibit interactions involving L-selectin. Also disclosed are methods of synthetic preparation and use.

### CROSS-REFERENCES TO RELATED APPLICATIONS

This application claims the benefit of U.S. Provisional Application No. 60/651,891, filed Feb. 10, 2005, which is incorporated herein by reference in entirety.

### STATEMENT ON U.S. FEDERALLY SPONSORED RESEARCH OR DEVELOPMENT

This invention was made with government support under NIH HL60464 and NIH HL57345 awarded by the National Institutes of Health. The government has certain rights in the invention.

### BACKGROUND OF THE INVENTION

Cell adhesion is a significant aspect of inflammatory processes which can involve Selectins, a family of cell-surface adhesion molecules. Selectin-induced leukocyte rolling on endothelial surfaces is an essential step in mediating events leading to inflammatory and cell-mediated immune responses. Characteristically, the adhesion cascade is facilitated by the interaction of selectins with O-glycosylated protein ligands that present sulfated derivatives of the tetrasaccharide sialyl Lewis x (Neu5Acα3Galβ4(Fucα3)GlcNAc-, sLe^{x}). Significant effort has been directed toward generating sLe^{x} mimetics in the form of small molecules, polymers, liposomes, and protein conjugates, as competitive inhibitors of selectin-mediated binding events. However, relatively weak affinity, susceptibility to hydrolytic cleavage, potential antigenicity, and short circulating half-life, in addition to the absence of a convenient synthetic route are acknowledged limitations of sLe^{x}-derivatized bioconjugates. Thus, motivation exists to develop alternative therapeutic oligosaccharide analogues capable of modifying cell adhesion events and inflammatory responses.

### SUMMARY OF THE INVENTION

In general the terms and phrases used herein have their art-recognized meaning, which can be found by reference to standard texts, journal references and contexts known to those skilled in the art. The following definitions are provided to clarify their specific use in the context of the invention.

When used herein, the term “branched glycopolymer” refers to a molecule having a plurality of arms, at least one multimeric component comprising a repeating monomeric unit, and a saccharide component. The molecule can have a core or backbone component, for example to serve as a connection point in a star-shaped, dendrimeric, comb-like, or other configuration. In some examples, a branched glycopolymer can be described as hyperbranched.

When used herein, the term “saccharide” refers to a sugar molecule and can include a monosaccharide and a polysaccharide. In a particular embodiment, the term can refer to a disaccharide such as lactose.

When used herein, the term “derivatized” refers to a modified molecule where one or more modifications can include the addition, deletion, or modification of a chemical component of an initial molecule. For example, a derivatized saccharide can be a sulfated lactose.

As used herein, the terms “inflammation,” “inflammatory disease,” and/or “inflammatory disorder” can refer to a disease or disorder characterized by, caused by, resulting from, or becoming affected by inflammation. Examples of inflammatory diseases or disorders include, but not limited to, acute and chronic inflammation disorders such as rheumatoid arthritis, osteoarthritis, inflammatory bowel diseases (including, but not limited to, Crohn's disease and ulcerative colitis), chronic obstructive pulmonary disorder (COPD), psoriasis, multiple sclerosis, asthma, diseases and disorders related to diabetic complications, fibrotic organ failure in organs such as lung, liver, kidney, vascular conditions, and other inflammatory complications of the cardiovascular system such as acute coronary syndrome.

When used herein, the term “alkyl” can include derivatized alkyl. Alkyl groups include straight-chain, branched and cyclic alkyl groups. Alkyl groups include those having from 1 to 20 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-20 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups optionally include substituted alkyl groups. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted.

The following abbreviations are applicable: PEO, poly(ethylene oxide); sLe^{x}, sialyl Lexis^{x}; AAA, abdominal aortic aneurysm.

As a summary of certain embodiments of the invention, a new class of high molecular weight polysulfated PEO dendrimer-like glycopolymer has been synthesized by a combination of arm-first and core-first methodologies followed by trichloroacetimidate glycosidation as a facile bioconjugation strategy. This is the first report to describe the synthesis and biological evaluation of complex branched PEO heparinoid mimics, which provide an easily accessible route to carbohydrate-based compounds with anti-inflammatory activity in vivo.

In embodiments, the invention provides compounds and methods relating to therapy for an inflammatory disease or other inflammatory condition.

In an embodiment, the invention provides a branched glycopolymer comprising the formula: L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core. In an embodiment, L is a sulfated lactose. In an embodiment, A comprises polyethylene oxide (PEO).

In an embodiment, X is the polymer core selected from the group consisting of alkyl and a phosphazene group. In other embodiments, the core X can be other moieties as would be understood in the art. In particular embodiments, a linker group can serve to connect the core X with the polymer arm component such as A. In particular embodiments, a linker group can serve to connect an arm A with the end group L or other end group as described herein; this linker can be the same or different than that which connects X to A in the event linkers are used in both places. In particular embodiments, a value for n is selected from the group consisting of n=2, 3, 4, and 12.

In an embodiment, m is from about 20 to about 50. In an embodiment, m is from about 30 to about 45. In an embodiment, m is from about 20 to about 50 and n is 3 or 4. In a particular embodiment, n=3 and m=40. In another particular embodiment, n=4 and m=30.

In an embodiment, X is alkyl. In an embodiment, X is neopentyl. In an embodiment, n=3 or 4.

In an embodiment, X is a phosphazene group. In an embodiment, the phosphazene group is a cyclolinear phosphazene. In an embodiment, the cyclolinear phosphazene is a cyclic trimer. In an embodiment, n is from about 6 to about 96. In an embodiment, n is from about 12 to about 48. In a preferred embodiment, n=12. In an embodiment, m=from about 2 to about 600. In an embodiment, n=12 and m=about 160 to about 170.

In an embodiment, the invention provides a branched glycopolymer wherein there is a single branch generation. In an embodiment, there is a plurality of branch generations. In an embodiment, there are two branch generations. In an embodiment, there is a first branch generation of six arms and a second branch generation of two arms from each of the first generation, and wherein the total number of arms is 12.

In an embodiment, the invention provides a method of preparing a branched glycopolymer composition of a structural formula represented by L-[Aₘ]ₙ-X; wherein L is a saccharide or a saccharide derivative; A is a polymeric arm comprising a poly(alkylene oxide) where m is a number of monomeric units; n is a number of branching arms where n=2 to about 100; and X is a polymer core. In particular embodiments, certain variations are as described herein. In an embodiment, the method comprises the steps of providing an imidated lactose donor group and performing a Schmidt glycosidation coupling. In an embodiment, the method comprises anionic polymerization using a core-first approach. In an embodiment, the method comprises synthesis of a first generation of PEO arms on a phosphazene core using an arm-first approach.

In an embodiment, the method comprises synthesis of a second generation of PEO arms, wherein the second generation is directly polymerized onto the first generation. In an embodiment, the method comprises synthesis of multiple further generations of PEO arms, wherein each further generation is directly polymerized onto the previous generation. In an embodiment, a total number of generations is from about 3 to about 8. In an embodiment, a total number of generations is two.

In an embodiment, the invention provides a method of modifying an inflammatory condition comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core. In particular embodiments, certain variations are as described herein.

In an embodiment, the invention provides a method of modifying a cell adhesion event comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core. In particular embodiments, certain variations are as described herein.

In an embodiment, the invention provides a method of modifying a selectin-mediated interaction comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core. In particular embodiments, certain variations are as described herein. In an embodiment, the selectin-mediated interaction involves a selectin selected from the group consisting of L-selectin, P-selectin, and E-selectin. In a particular embodiment, the selectin-mediated interaction involves L-selectin. In a preferred embodiment, L is the lactose derivative which is a sulfated lactose.

In an embodiment, the method of modifying an inflammatory condition, a cell adhesion event, and/or an selectin-mediated interaction can occur in vivo, in vitro, or ex vivo. In a particular embodiment, said modifying occurs in vivo. In a particular embodiment, said modifying occurs in vitro. In a more particular embodiment, said in vitro modifying occurs under hemodynamic flow conditions.

In an embodiment, the invention provides a method of therapy for abdominal aortic aneurysm comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

In an embodiment, the invention provides a medical device treated with a compound of formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core; wherein said treated device has at least a portion of a surface that is capable of contacting a patient. In an embodiment, the medical device is selected from the group consisting of a stent, embolization coil, vascular graft, or other biomedical device capable of exposure to a patient.

In an embodiment, the invention provides a medical device, cell, tissue, or organ further comprising a film, gel, or other coating with a compound of the invention.

In an embodiment, the invention provides a branched glycopolymer comprising the formula: L-[A2ₘ₂]ₙ₂-[A1ₘ₁]ₙ₁-X; wherein L is lactose or a lactose derivative; A1 and A2 are polymeric arms comprising a poly(alkylene oxide) where m1 and m2 are numbers of monomeric units; n1 and n2 are numbers of branching arms where n1=2 to about 100 and n2=2 to about 100; and X is a polymer core. In an embodiment, m1 is from about 2 to about 400 and m2 is from about 2 to about 100. In an embodiment, m1 is from about 100 to about 150 and m2 is from about 10 to about 25. In a particular embodiment which is believed to correspond approximately to compound SR-12 (also referred to as 3c), A1 and A2 are PEO; m1 is about 144 by calculation and n1=6; m2 is about 22 by calculation and n2=2. In particular embodiments, certain variations are as described herein.

In an embodiment, the invention provides a branched glycopolymer wherein A1 and A2 are each poly(ethylene oxide); m1 is from about 100 to about 150; n1 is 6; m2 is from about 10 to about 30; n2 is 2, and L is selected from the group consisting of lactose, sulfated lactose, and other derivatized lactose.

In an embodiment, the invention provides a branched glycopolymer comprising the formula: S-[Aₘ₂]ₙ₂-[Aₘ₁]ₙ₁-X; wherein S is a saccharide or a saccharide derivative; A is a polymeric arm comprising a poly(alkylene oxide) where m1 and m2 are numbers of monomeric units; n1 and n2 are numbers of branching arms where n1=2 to about 100 and n2=2 to about 100; and X is a polymer core. In particular embodiments, certain variations are as described herein.

In an embodiment, the invention provides a method of functionalizing a PEO-based polymer.

In particular embodiments, the invention provides compounds designated 1a, 1b, 1c (also referred to as SR-3); 2a, 2b, 2c (also referred to as SR-12); and 3a, 3b, 3c (also referred to as SR-12); for chemical structure formulas, see, e.g., FIG. 1B, FIG. 1C, FIG. 2, etc.

In embodiments, the invention provides methods and compounds in connection with exposure of substances and devices to a mammalian body, including a human body. The blood contacting materials, prostheses and other implantable materials and devices, surface coated according to the methods of the present invention, can include, without limitation, vascular grafts, embolization coils, shunts, stents, small diameter (about 4 to about 6 mm inner diameter), dialysis tubing, membranes and hollow fiber systems, membrane oxygenators, artificial heart valves, left ventricular assist devices, other biomedical devices capable of implantation, and medical diagnostic devices as well as biological material for exposure to/implantation into a patient, for example, heterograft tissues including but not limited to porcine heart valves and bovine carotid vascular grafts. Surface coating of a blood contacting organ such as an artificial heart, lung, kidney or liver is within the scope of the present invention.

In embodiments, compounds of the invention are prepared in a pharmaceutical formulation as understood in the art, for example using compatible solutions and/or excipients. Pharmaceutical salts are prepared as would be understood in the art. In an embodiment, the invention provides a pharmaceutically acceptable salt of a compound of the invention. In an embodiment, the invention provides a pharmaceutical formulation of a compound of the invention.

In embodiments, the invention provides a method of delivering a compound comprising the step of introducing, applying, or otherwise exposing the compound to a subject. In an embodiment, the compound is applied via intravenous, subcutaneous, intraosseous, intravitreal, intranasal, per os (oral), intraocular, or other appropriate route as known in the art.

In embodiments, the invention provides compounds and methods for construction of sulfated polyanionic glycodendritic PEO bioconjugates as glycosaminoglycan mimicking molecules for therapeutic intervention in selectin inhibition and human inflammatory disorders.

In embodiments, the invention provides therapeutic oligosaccharide analogues that are selectin-binding antagonists which exhibit multiple and cooperative receptor binding properties.

In embodiments, compounds of the invention can have prodrug forms. Prodrugs of the compounds of the invention are useful in the methods of this invention. Any compound that will be converted in vivo to provide a biologically, pharmaceutically or therapeutically active form of a compound of the invention is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et. al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

In embodiments, the invention provides methods of regulating leukocyte infiltration by selective inhibition of L-selectin.

The invention substantially as herein described and illustrated is provided. The invention provides, inter alia, a new compound, substantially as herein described; a new use of a compound, substantially as herein described; a substance or composition for a new use in a method of treatment, substantially as herein described; and/or a new process for preparing a compound, substantially as herein described.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE FIGURES

FIG. 1A illustrates the potential interaction of a cell surface receptor with a molecule of the selectin family and a blockade of the interaction with a polyvalent branched glycoconjugate. FIG. 1B illustrates glycosidation of terminal hydroxyls using lactose imidate (A). The 12-arm substrate is not shown but is also capable of reacting similarly to yield an analogous 12-arm product.

FIG. 2 illustrates saccharide-functionalized PEO star and “dendrimer-like” polymers as selectin ligands.

FIG. 3 illustrates results of an ¹H NMR (600 MHz) spectrum of 3a. Inset shows the MALDI-TOF spectra of the original hydroxyl-terminated polymer and 3a.

FIG. 4 illustrates results of neutrophil (A) and macrophage (B) content in thioglycollate-induced peritoneal inflammation (n=6). 1c/2c did not exhibit substantial in vivo activity. Each bar represents the average value +/−SD.

FIG. 5 illustrates results of U937 cell adhesion to immobilized (A) L-selectin, (B) P-selectin. Neither heparin nor 3c exhibited substantial inhibitory activity toward E-selectin (C). Inhibition was not observed in the absence of test compound or heparin. Data represent means of at least n=3 (SD <10%).

FIG. 6 illustrates results in vivo regarding abdominal aortic aneurysm.

FIG. 7 illustrates early steps in a synthetic scheme for generation of 3 and 4 star arm PEO polymers with hydroxy terminal end groups.

FIG. 8 illustrates a synthetic scheme for generation of 3 and 4 star arm PEO polymers with functionalized end groups having sulfated lactose units.

FIG. 9. ¹H NMR of 1a

FIG. 10. ¹³C NMR of 1a

FIG. 11. ¹H NMR of 1b

FIG. 12. ¹³C NMR of 1b

FIG. 13. ¹H NMR of 1c

FIG. 14. ¹³C NMR of 1c

FIG. 15. ¹H NMR of 2a

FIG. 16. ¹³C NMR of 2a

FIG. 17. ¹H NMR of 2b

FIG. 18. ¹³C NMR of 2b

FIG. 19. ¹H NMR of 2c

FIG. 20. ¹³C NMR of 2c

FIG. 21. ¹H NMR of 3a

FIG. 22. ¹³C NMR of 3a

FIG. 23. ¹³C NMR of 3b

FIG. 24. ¹H NMR of 3c

FIG. 26. ¹³C NMR of 3c

FIG. 26. Representative FTIR spectra of non-sulfated PEO-glycodendron 2b and sulfated glycodendron 2c. After sulfation, new peaks appeared at v 1248 cm⁻¹ and 810 cm⁻¹ which are characteristic of sulfoxide (S═O) and a C—O—S stretching bond. A similar pattern was observed in case of the 3-arm glycoderivative 1b/1c.

FIG. 27. SDS-PAGE analysis (tris-tricine 16.5%) of sulfated 3-arm (1b, 1c) and 4-arm (2b, 2c) glycopolymers. A: Staining sulfated glycopolymers. The gel was stained using Toluidine blue (0.2 g of Toluidine Blue O+50 mL of EtOH+49 mL H₂O+1 mL AcOH) for 30 min in a shaker. Destaining (50 mL of EtOH+49 mL H₂O+1 mL AcOH) was then performed. The gel was also stained with Coomassie blue in order to identify molecular weight markers. B: Staining polyethylene oxide components. The gel was incubated in a 5% BaCl₂ solution for 5 min and then washed with water. The gel was then stained with 12 solution and destained with water.

FIG. 28. MALDI-TOF and GPC profiles for 3-arm glycopolymers

FIG. 29. MALDI-TOF and GPC profiles for 4-arm glycopolymers

FIG. 30. MALDI-TOF data for 3a, 3b and SELDI-TOF profile of 3c.

### DETAILED DESCRIPTION OF THE INVENTION

The invention may be further understood by the following non-limiting examples.

### EXAMPLE 1

### Hyperbranched PEO Glycopolymers Exhibit Anti-Inflammatory Properties In Vivo

Introduction. The simultaneous presentation of saccharide epitopes on an appropriate macromolecular scaffold creates a multivalent display that amplifies the affinity of glycoside-mediated receptor targeting. In this regard, branched poly(ethylene oxide) (PEO) polymers can provide useful scaffolds for in vivo blockade of selectin binding due to their defined molecular architecture, hydrophilicity, and availability of multiple surface reactive sites. Moreover, the branched polymer structure also provides a mechanism for controlling accessibility, mobility, density, and supramolecular organization of pendant sugar epitopes, as additional elements that may facilitate the design of optimal selectin-binding antagonists with defined circulating half-life. Herein, we report a simple strategy for the synthesis of 1st and 2nd generation dendrimer-like PEO glycopolymers bearing sulfated β-lactose as potential L-selectin inhibitors.

Most current glycoconjugation strategies describe the use of an aliphatic or aromatic aglycone linker that carries an amine, thiol, thiourea, acid, or active ester as the reactive moiety. Significantly, by using imidated lactose donors and a Schmidt glycosidation coupling protocol, protecting group manipulations were minimized in the process of glycopolymer synthesis.

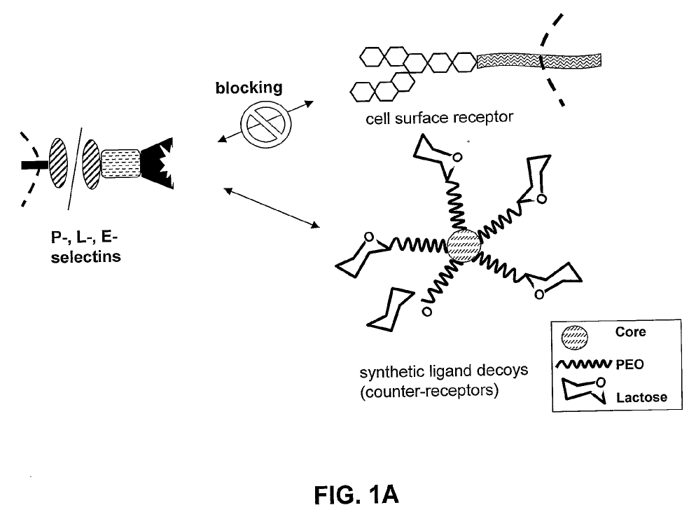

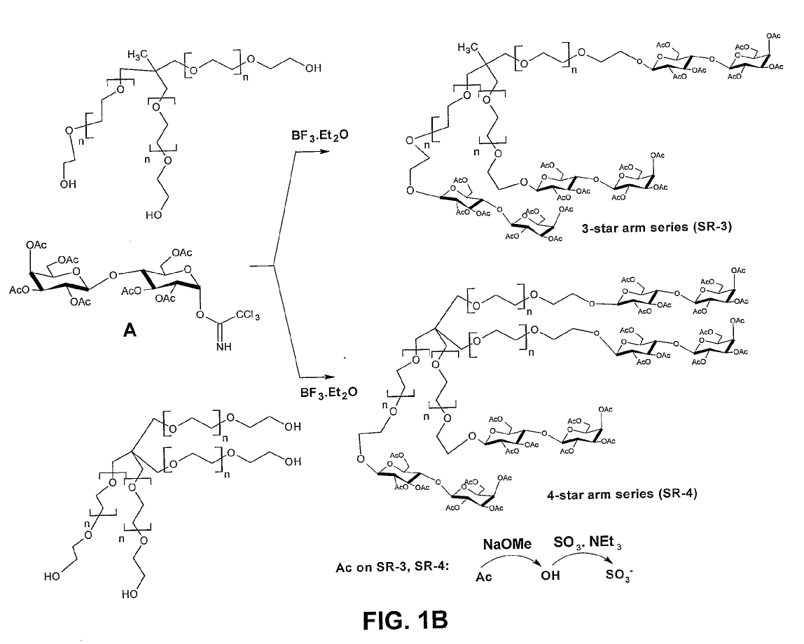

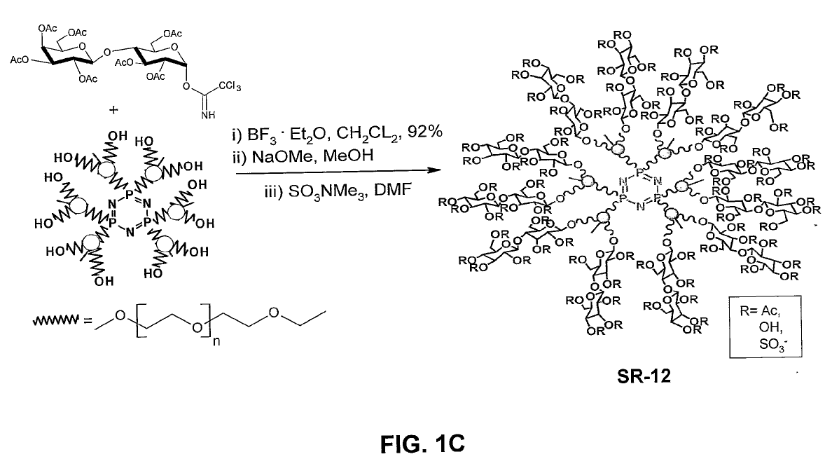

Three (Mn ˜5200 mu) and four arm (Mn ˜5100 mu) PEO stars (1st generation) carrying hydroxy end groups were synthesized by anionic polymerization using “core-first” methodology (Hou et al. 2003, Macromolecules 36:3874-3881; Angot et al. 2000, Macromolecules 33:5418-5424). In addition, a dendrimer-like 2^{nd} generation PEO star polymer (12 arm) was synthesized based on a phosphazene core. This polymer consisted of a 1st generation of six PEO arms, produced by an “arm-first” strategy onto the phosphazene core, followed by a 2nd generation of 12 hydroxyterminated PEO branches polymerized directly onto the original six arm core (Mn ˜52 kD) (Hou et al. 2003, Polymer 44:5067-5074). β-Lactose octaacetate was selectively brominated at the anomeric center and subsequently activated to the imidate donor (A) as indicated in FIG. 1B.

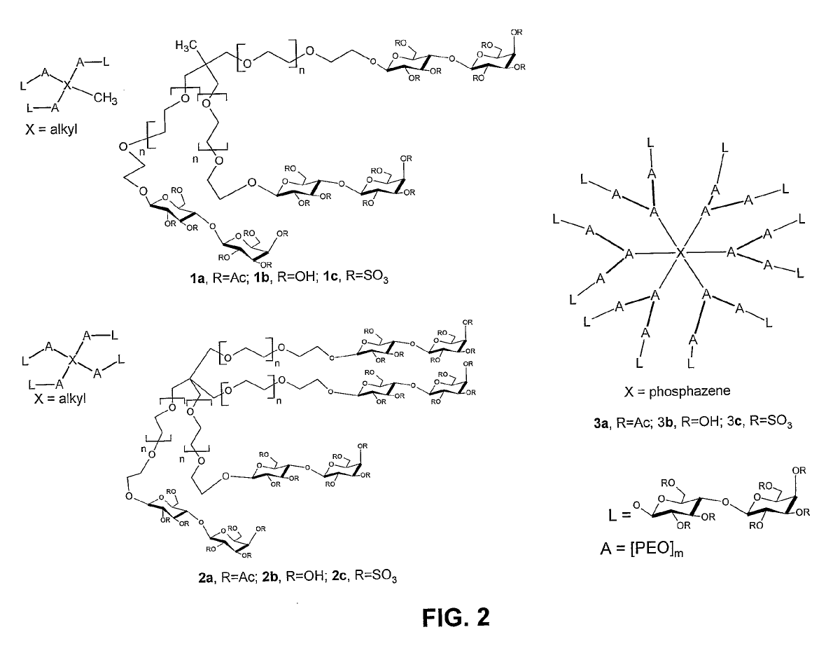

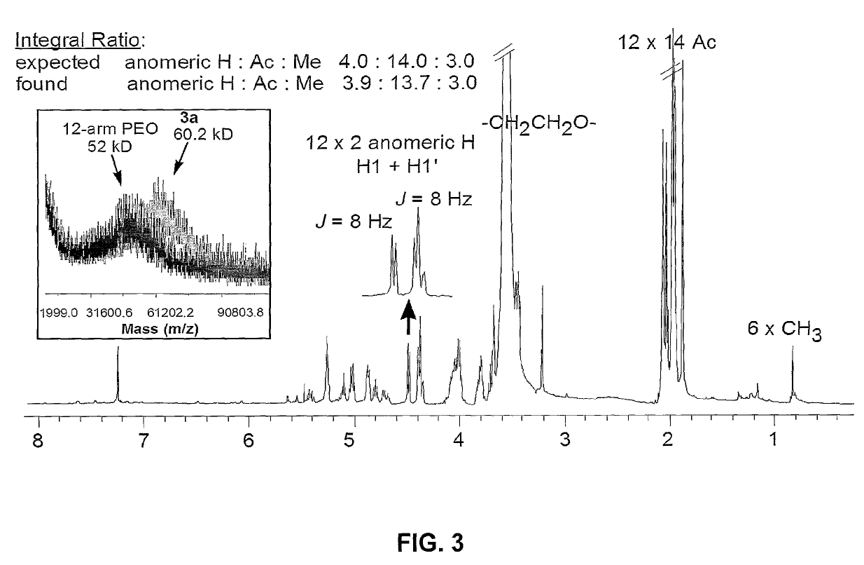

Glycosylations of hydroxy-terminated PEO star and dendrimer-like polymers by trichloroacetimidate glycosidation methodology, using BF₃.OEt2 as a Lewis acid activator resulted in the covalent attachment of acetyl-protected lactose residues in high yield. Zemplen conditions for deacetylation (NaOMe/MeOH) followed by lactose sulfation using an excess of SO₃.trimethylamine complex furnished target PEO glycoclusters carrying terminal sulfated oligosaccharides 1c, 2c, and 3c (see FIG. 2). The efficiency, homogeneity, and degree of ligand (lactose) loading on the PEO polymers were estimated by ¹H NMR spectroscopy, as well as by mass estimates obtained by MALDI-TOF and laser light scattering. Moreover, FTIR and SDS-PAGE analysis provided additional evidence of sulfated lactose units.

Specifically, the relative intensities of the anomeric H:Ac:CH₃ signal ratio of 6:20.9:3 for the three arm (1a) derivative and an integration ratio of 7.95:27.97:8 for the four arm glycocluster 2a indicated complete glycosylation of the hydroxyl groups on the parent PEO precursor. The increase in molecular weight was further corroborated using MALDI-TOF (1a: 6899 mu, 2a: 7524 mu) and LLS measurements, confirming quantitative functionalization. Likewise, an NMR integration ratio of 3.9:13.7:3, as well as MALDI-TOF, demonstrated a high degree of lactose conjugation (>95%) onto the dendritic PEO scaffold of the 12-arm, 2^{nd} generation, branched compound. Subsequent deprotection followed by sulfation produced a highly charged sulfated glycodendron 3c (observed SELDI-TOF: 61.8 kD, expected value ˜62 kD).

Heparin can exhibit anti-inflammatory properties by mediating blockade of L- and P-selectins via sulfate-dependent interactions. In vitro observations have reported that sulfated esters can promote selectin binding when appropriately oriented on a lactose core. For example, a sulfated lactose derivative (6,6′-disulfo lactose), lacking fucose and sialic acid residues, was superior to sLe^{x} as an in vitro inhibitor of L-selectin binding to GlyCAM-1. See references 8(a, b, c).

We explored the capacity of sulfated branched star and dendrimer-like glycopolymers to limit inflammatory responses in vivo via presumed selectin-dependent blockade on the theory that selectin-glycoligand binding can be greatly amplified through multivalent presentation of oligosaccharide determinants. We tested this approach using our compounds in the context of an inflammatory condition, peritonitis.

Acute inflammation was induced in a mouse model by thioglycollate injection into the peritoneal cavity. Potency was valence-dependent with 1c/2c exhibiting little activity, while 3c (0.5 mg/mouse IV) dramatically reduced neutrophil and macrophage recruitment by 86 and 60%, respectively (FIG. 4, p<0.05). Although heparin inhibited inflammatory cell recruitment to a similar degree, we believe that concurrent anticoagulant effects pose practical limitations on heparin's clinical applicability. In contrast, 3c does not exhibit substantial anti-thrombin activity (data not shown).

### Methods.

Thioglycollate induced murine model of peritoneal inflammation. Mice (C57BL, male, 6-8 weeks old) were injected intraperitoneally with 2 mL of 3% thioglycollate broth. Five minutes later, animals received intravenous injections of 0.2 mL sterile pyrogen-free saline (S) with and without heparin (H) or analogs (3c) (0.5 mg/mouse with 0.2 ml saline). Mice were sacrificed after 3 hours and peritoneal cells harvested by lavage with 8 mL of ice-cold phosphate buffered saline (PBS) containing 3 mM EDTA. Peritoneal cells were counted and cells stained for 30 minutes at 4° C. with FITC-conjugated rat anti-mouse Gr-1 mAb diluted in PBS containing 2.5% FBS. After washing three times with PBS, FACScan analysis was performed as described by Wang et al. (2002) and was used to sort, count and analyze the percentage of neutrophils and macrophages which were converted to the cell number using the formula N/M=Total cell number X % of NIM, respectively (N:Neutrophil; M: Macrophages). The cells were gated (FACS) expressing a high level of Gr-1 antigen (N) and Mac-1 (M). (See additional ref: Lagasse; E., Weissman; I. J. Immunological Meth. 1996, 197, 139-150). Statistical differences among groups were analyzed by ANOVA method using StatView software.

### EXAMPLE 2

### Demonstration of Functional Activity in the Inhibition of Cell Adhesion

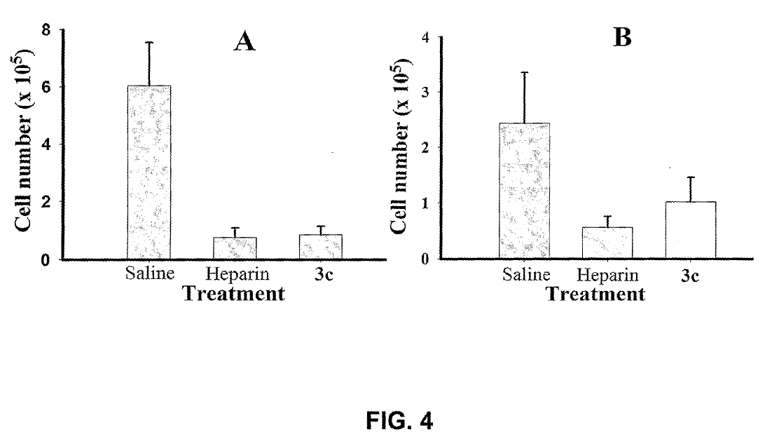

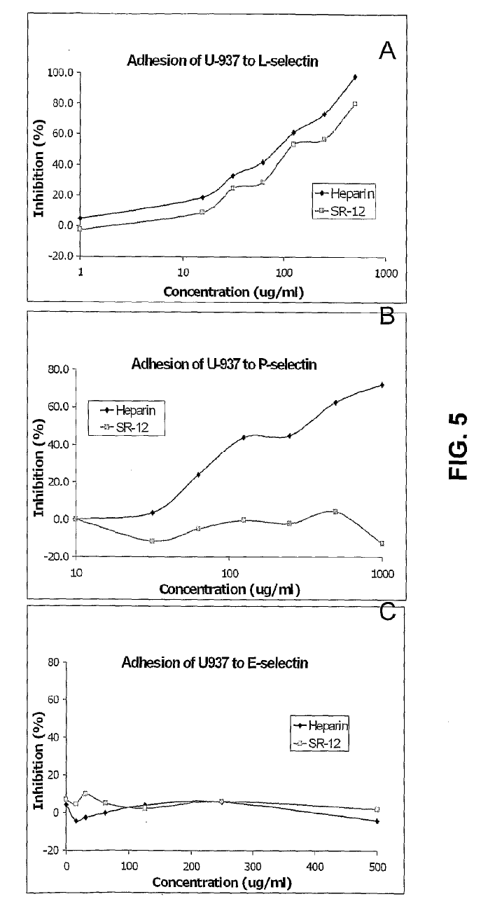

The ability of 3c to inhibit the adhesion of U937 cells to immobilized selectins was examined to determine whether the observed in vivo effect was mediated by presumed selectin blockade. 3c selectively blocked the adhesion of U937 cells to L-selectin in a dose-dependent manner (IC₅₀=2.4 nM), but did not exhibit anti-P-selectin or E-selectin activity. See FIG. 5. Therefore, 3c is among the most potent L-selectin inhibitors yet reported (see references 2a, 8-10).

A partial explanation for this substantial biological activity can be that increased biological activity is a consequence of ligand presentation by dendrimeric scaffolds when compared to linear counterparts. Despite significant anti-L-selectin activity, the observed in vivo activity was surprising since it has been recently reported that heparin's anti-inflammatory activity in vivo is critically dependent on its ability to inhibit both L- and P-selectin mediated inflammatory cell adhesion (Wang et al., 2002). Indeed, a compound acting solely as a selective inhibitor of L-selectin is not anticipated to block in vivo leukocyte infiltration so completely. Thus, it is likely that 3c, like heparin, blocks chemokine binding to the endothelium, which would further limit leukocyte extravasation.

### Methods.

U937 cell binding to immobilized E-, L- or P-selectin. The ability of test compounds to competitively inhibit the adhesion of U937 cells to immobilized P-, L- or E-selectins was examined by coating each well of a 96-well plate at 4° C. overnight with 8 μg/mL of Protein A in 50 mM carbonate buffer, pH 9.4. After blocking the plate with 10% BSA in PBS, P-selectin (0.05 μg/well), L-selectin (4 μg/well) or E-selectin (1 μg/well) chimeras were added. After 1 hr, wells were washed with blocking solution. U937 cells were grown in RPMI-1640 medium (containing 10% fetal bovine serum (FBS), 100 U/ml penicillin and 100 μg/ml streptomycin in an atmosphere of 5% CO₂ in air and 100% relative humidity. The cells were fluorescently labeled with 10 μM calcein AM in RPMI 1640 medium containing 2.5% FBS for 30 min at 37° C. The cells were collected by low speed centrifugation, washed three times with RPMI 1640 medium, and resuspended at a density of 2×10⁶ cells/mL in medium without FBS. Heparin and test compounds, or 10 mM sodium EDTA (negative control) were added at 50 μL/well, and then the fluorescently labeled U937 cell suspension (50 μL/well) was added and incubated for 30 min at room temperature. Non-adherent cells were removed by rinsing the plates three times with PBS, and the number of adherent cells was quantified by measuring the fluorescence intensity at 485 nm after cell with 2% Triton X-100 in 0.1 M Tris-HCl, pH 9.5. Raw data were converted to relative fluorescence intensity (RFI) for comparative purposes.

### EXAMPLE 3

### Synthetic Procedures and Analysis

### Experimental

General Methods: All chemicals were reagent grade and used as supplied unless otherwise noted. Solvents were dried and distilled before use. Molecular sieves were activated at 350° C. for 3 h in vacuo. Dichloromethane was distilled from CaH₂ and stored over 4 Å molecular sieves. All of the reactions were performed with oven-dried glassware under argon atmosphere and monitored by thin layer chromatography (TLC) on 250-μm Al pre-coated silica gel plates. Detection was performed by examination under UV light (254 nm) and by charring with 10% sulfuric acid in water. Flash chromatography was performed on silica gel (mesh 200-425) with the appropriate solvents. Size exclusion chromatography was performed with water as eluant. Extracts were concentrated under reduced pressure at <45° C. (water bath). ¹H NMR and ¹³C NMR spectra were recorded on 300 MHz, 400 MHz or 600 MHz NMR spectrometers. For ¹H NMR and ¹³C NMR spectra recorded in CDCl₃, CD₃OD, D₂O and DMSO chemical shifts (δ, delta) are given in ppm relative to solvent peaks. Coupling constants (J) are reported in Hertz (Hz). MALDI-TOF mass spectrometry data was performed using 2,5-dihydroxybenzoic acid as the matrix. Surface enhanced laser desorption/ionization time-of-flight mass spectrometry (SELDI-TOF MS) was carried out on sulfated 12-stararm dendrimer 3c using sinapinic acid and “CDC oligo” matrix obtained from Centre for Disease control. BSA was used as an external standard for SELDI-TOF experiments. Sample preparation for SDS-Page gel analysis was carried out by diluting 25 μL of sample solution with 25 μL stock solution of Laemmli buffer solution (95 μL of Laemmli buffer solution+95 μL of mercaptoethanol). A total amount of 50 μL was loaded on the gel and electrophoresis samples run in a 10×Tris/Glycine/SDS buffer solution.

General procedure for synthesis of poly(ethylene oxide) glycopolymers (1a, 2a, 3a).

Acetylated lactose imidate (1.5 eq/each OH) was added to an ice-cooled solution of OH-terminated poly(ethylene oxide) dendrimer-like polymers containing 3, 4, 12 terminal arms dissolved in anhydrous CH₂Cl₂. The Lewis acid BF₃.Et₂O (5 eq.) was added to the reaction mixture and stirred at 0° C. for 1.0 h followed by stirring at room temperature for 16 h. The reaction was quenched with diisopropylamine and concentrated and subjected to silica gel column chromatography using CHCl₃:MeOH as eluent (0%→10%). The product was obtained as a white solid. All acetyl protected glycopolymers 1a, 2a, 3a were soluble in water.

General procedure for deacetylation to give 1b, 2b, 3b. NaOMe (1.5 eq/Ac group) was added via syringe to a solution of acetylated glycopolymers in dry MeOH and the reaction mixture stirred at room temperature for 5 h. On completion, as determined by the disappearance of the starting compound on TLC, the reaction mixture was passed through an H⁺ ion exchange column, filtered, and washed five times with MeOH. The collected MeOH fractions were pooled and concentrated to give an off-white residue, which was purified using a gel filtration column with water as eluant and freeze dried.

General procedure for sulfation of glycopolymers 1c, 2c, 3c. The sulfur trioxide-trimethylamine complex was extensively washed with water to remove large amounts of trapped H₂SO₄, followed by three washes each with ethanol and CH₂Cl₂, filtration and vacuum drying. The SO₃.NMe₃ complex was added to a solution of deprotected glycopolymers (1b, 2b, 3b) in dry DMF in molar excess (7 equivalents per hydroxyl group) and the reaction mixture stirred at 60° C. for 3 days. An five equivalents of SO₃.NMe₃ complex was then added and stirring continued at 60° C. for an additional 3 days. The reaction mixture was then filtered and the residue washed thoroughly with MeOH/water (50:50). The reaction solution was concentrated and passed through a gel filtration column using MeOH/water as the eluant. Products 3b to 3c were dialyzed for 3 days, freeze dried, and then column purified. Appropriate fractions were pooled and lyophilized to afford the sulfated glycopolymers as amorphous white solids. All compounds were characterized using ¹H and ¹³C NMR, MALDI-TOF, laser light scattering, and SDS-PAGE gel electrophoresis.

Spectral Data of 1a. ¹H NMR (CDCl₃, 400 MHz): 5.30 (d, 3H, J=2.8 Hz), 5.14 (t, 3H, J=9.6 Hz, J=9.2 Hz), 5.08-5.03 (m, 3H), 4.92 (dd, 3H, J=3.2 Hz, J=3.6 Hz), 4.85 (m, 3H), 4.52 (d, 3H, J=8.0 Hz), 4.44 (d, 3H, J=7.6 Hz), 4.2-4.0 (m, 12H), 3.92-3.71 (m, 6H), 3.68-3.34 (m, PEO backbone), 3.22 (s, 6H, —CH₂—O—), 2.08-1.91 (7×3 COCH₃), 0.89 (s, 3H, CH₃).

¹³C NMR (CDCl₃, 400 MHz): 164.1 (COCH₃), 164.0 (COCH₃), 163.8 (COCH₃), 163.7 (COCH₃), 163.4 (COCH₃), 163.3 (COCH₃), 162.7 (COCH₃), 94.7 (C-1′), 94.2 (C-1), 69.9, 67.5, 66.4, 66.2, 65.3, 64.7, 64.6, 64.2, 64.0, 63.9, 63.8, 62.7, 60.2, 59.5, 55.6, 55.3, 54.4, 34.6, 23.3, 14.5, 14.4, 14.3, 14.2, 14.1, 10.9. MALDI-TOF: found 6899.8299 (expected 7050).

Spectral Data of 1b (deprotected) ¹H NMR (D₂O, 400 MHz): 4.35 (d, 3H, J=8.4 Hz), 4.28 (d, 3H, J=7.6 Hz), 3.90-3.34 (m, —OCH₂CH₂O skeleton), 3.25 (s, 6H, —CH₂—O), 3.16 (t, J=8.0 Hz, J=8.4 Hz), 0.87 (s, 3H, CH₃). ¹³C NMR (D₂O, 400 MHz): 103.1, 102.2, 78.5, 75.5, 74.9, 74.4, 73.6, 72.9, 72.6, 71.9, 71.1, 70.7, 69.7, 69.3, 68.8, 68.7, 61.2, 60.5, 60.2, 40.6, 17.0. MALDI-TOF: found 5909.2649 (expected 6100).

Spectral Data of 1c (sulfated) ¹H NMR (D₂O, 600 MHz): 4.94-4.92 (m, sugar ring protons), 4.4-4.0 (m, sugar ring protons), 3.93-3.42 (m, OCH₂CH₂O—), 3.26 (s, 6H), 3.1-3.04 (m, sugar ring protons), 0.78 (s, CH₃). ¹³C NMR (D₂O, 400 MHz): 94.8 (C-1′), 93.2 (C-1), 71.1, 70.8, 69.4, 69.0, 68.7, 68.4, 67.6, 67.1, 65.5, 64.1, 63.2, 62.9, 62.4, 62.2, 61.2, 60.8, 59.9, 34.1, 10.5.

Spectral Data of 2a ¹H NMR (CDCl₃, 600 MHz): 5.05 (t, 4H, J=10.2 Hz), 4.96 (t, 4H, J=8.4 Hz, J=8.4 Hz), 4.82 (m, 4H), 4.74 (t, 4H, J=7.8 Hz), 4.4 (d, 4H, J=8.4 Hz), 4.35 (d, 4H, J=7.8 Hz), 4.34 (d, 4H, partly merged with H-1), 4.1-3.92 (m, 16H), 3.8-3.72 (m, 8H), 3.7-3.38 (m, —OCH₂CH₂O— skeleton), 3.28 (s, 8H, CH₂—O), 2.01-1.82 (7×4 COCH₃). ¹³C NMR (CDCl₃, 600 MHz): 163.9 (COCH₃), 163.8 (COCH₃), 163.6 (COCH₃), 163.5 (COCH₃), 163.2 (COCH₃), 163.1 (COCH₃), 162.6 (COCH₃), 94.6 (C-1′), 94.2 (C-1), 69.8, 66.4, 66.2, 65.2, 64.6, 64.1, 63.9, 63.8, 63.5, 62.6, 60.1, 55.6, 54.4, 47.2, 39.4, 25.1, 16.2, 14.4, 14.3, 14.2, 14.1, 14.0. MALDI-TOF: found 7524.18 (expected 7500).

Spectral Data of 2b (deprotected) ¹H NMR (D₂O, 600 MHz): 4.34 (d, 4H, J=7.8 Hz), 4.26 (d, 4H, J=7.8 Hz), 3.89-3.87 (m, ring protons), 3.81-3.78 (dd, ring protons, J=2.4 Hz), 3.75 (d, ring protons, J=3.0 Hz), 3.7-3.37 (m, —OCH₂CH₂O— skeleton), 3.32 (s, 8H, —OCH₂), 3.18-3.15 (m, ring protons). ¹³C NMR (D₂O, 600 MHz): 103.1, 102.3, 78.5, 75.5, 74.9, 74.4, 72.9, 72.6, 71.1, 70.7, 69.7, 68.8, 68.7, 61.2, 60.2, 45.2. MALDI-TOF: found 6356.7333 (expected 6300)

Spectral Data of 2c (sulfated) ¹H NMR (D₂O, 600 MHz): 4.96 (d, 4H, J=2.4 Hz), 4.9 (d, 4H, J=6.0 Hz), 4.64-4.62 (m, ring protons), 4.59-4.57 (m, ring protons), 4.38 (dd, ring protons, J=2.4 Hz, J=3.0 Hz), 4.34 (t, ring protons, J=5.4 Hz, J=4.2 Hz), 4.31 (dd, ring protons, J=5.4 Hz, J=4.8 Hz), 4.24 (d, ring protons, J=8.4 Hz), 4.21 (d, ring protons, J=7.8 Hz), 4.12-4.05 (m, ring protons), 3.97-3.85 (m, ring protons), 3.72-3.46 (m, —OCH₂CH₂O— skeleton), 3.36 (s, 8H, CH₂O—), 3.08 (m, sugar ring protons). ¹³C NMR (D₂O, 400 MHz): 94.7, 93.3, 71.2, 70.8, 69.4, 69.1, 68.7, 68.5, 67.2, 65.4, 64.2, 63.8, 63.2, 62.6, 62.3, 60.5, 59.9, 45.8.

Spectral Data of 3a ¹H NMR (CDCl₃, 600 MHz): 5.61-5.38 (m, ring protons), 5.26-5.23 (m, ring protons), 5.11-4.99 (m, ring protons), 4.87-4.66 (m, ring protons), 4.47-4.36 (m, 24 anomeric H), 4.2-3.92 (m, ring protons), 3.66-3.44 (m, PEO skeleton), 3.42 (s, PEO skeleton), 2.07-1.87 (7×12 COCH₃), 0.9 (s, 18H, CH₃). ¹³C NMR (CDCl₃, 600 MHz): 163.9, 163.7, 163.6, 163.3, 163.2, 163.1, 162.6, 94.7, 94.4, 94.2, 83.5, 70.1, 69.9, 68.7, 67.4, 66.4, 66.2, 65.2, 60.1, 64.7, 64.6, 64.1, 63.9, 63.8, 63.6, 63.4, 62.7, 62.5, 61.4, 60.2, 55.6, 54.4, 34.2, 32.1, 14.5, 14.4, 14.2, 14.1, 10.8. MALDI-TOF: found 60254 (expected 59500).

Spectral Data of 3b (deprotected) ¹H NMR (D₂O, 600 MHz): 4.54-4.48 (m, ring protons), 4.37-4.26 (d, ring protons), 4.16-3.88 (m, ring protons), 3.82-3.32 (m, PEO skeleton), 3.12 (s, PEO skeleton), 3.08-3.06 (m, ring protons), 0.81 (s, CH₃). ¹³C NMR (D₂O, 600 MHz): 96.5, 95.7, 72.1, 71.9, 71.8, 70.6, 68.9, 68.3, 67.9, 67.8, 67.4, 67.1, 66.4, 66.1, 65.1, 64.7, 64.5, 64.1, 63.8, 63.2, 62.5, 62.3, 62.1, 61.3, 54.6, 53.6, 34.1, 10.4. MALDI-TOF: found 56132.266 (expected approx 56 kD).

Spectral Data of 3c ¹H NMR (D₂O, 600 MHz): 4.96 (s, ring protons), 4.84 (d, ring protons, J=6.0 Hz), 4.54-4.53 (m, ring protons), 4.36-4.28 (m, ring protons), 4.24-4.21 (m, ring protons), 4.19 (d, ring protons), 4.06-3.83 (m, ring protons), 3.71-3.38 (m, PEO backbone), 3.52 (s, PEO backbone), 1.23-1.02 (m), 0.82 (s, CH₃).

¹³C NMR (D₂O, 600 MHz): 94.6, 93.1, 71.3, 70.7, 69.3, 69.0, 68.7, 68.5, 67.1, 66.9, 65.3, 64.2, 63.762.1, 61.4, 60.3, 59.8, 34.1, 10.4. SELDI-TOF: found 61852.9386 (expected approx 62.6 kD).

### EXAMPLE 4

### Demonstration of Functional Activity in the In Vivo Context of Abdominal Aortic Aneurysm

Abdominal aortic aneurysms (AAAs) affect 2-9% of the population and are the 10th leading cause of death in white men over age 65. The disease can be characterized by thinning of the extracellular matrix in the aortic media with associated destruction of elastin, loss of smooth muscle cells, and transmural infiltration of inflammatory cells. Certain molecules such as MMPs, chemokines, proinflammatory mediators can be upregulated. L-selectin dependant leukocyte-endothelial cell interactions can play an important role in the genesis of the inflammatory response, which leads to aortic aneurysm formation.

We have synthesized a multivalent glycodendrimer, designated SR-12, whose anti-inflammatory activity is mediated, at least in part, by an anti-L-selectin effect (IC₅₀ 2.4 nM). We hypothesize that L-selectin blockade via multivalent, glycodendrimers will suppress the formation and/or growth of abdominal aortic aneurysms by inhibition of leukocyte recruitment to the vascular wall.

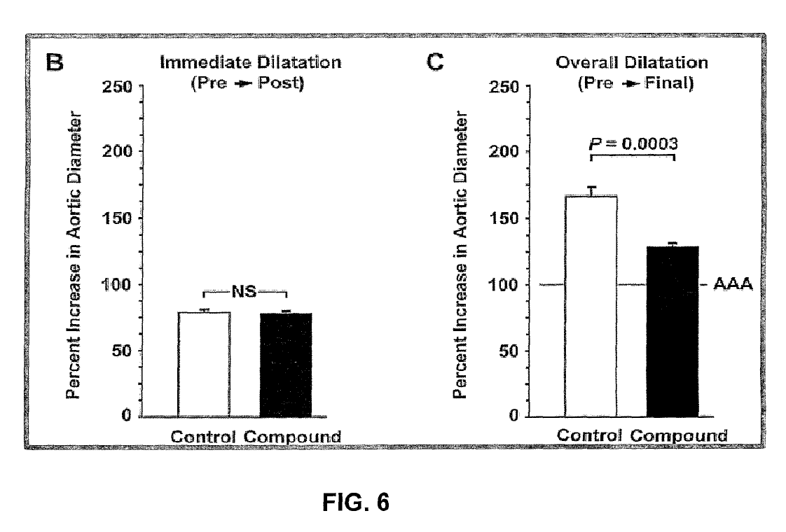

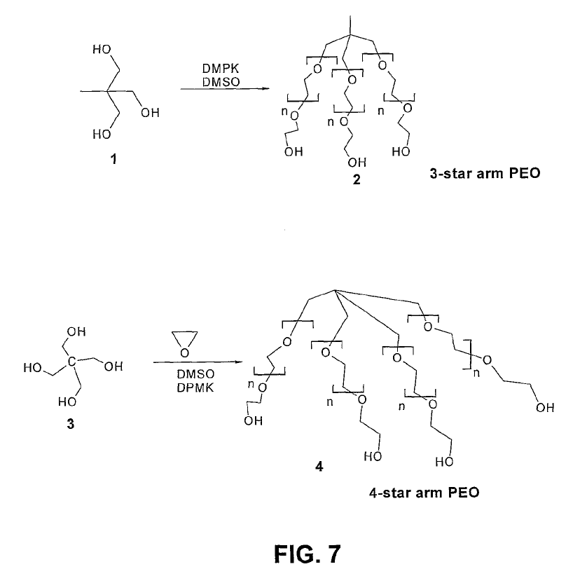

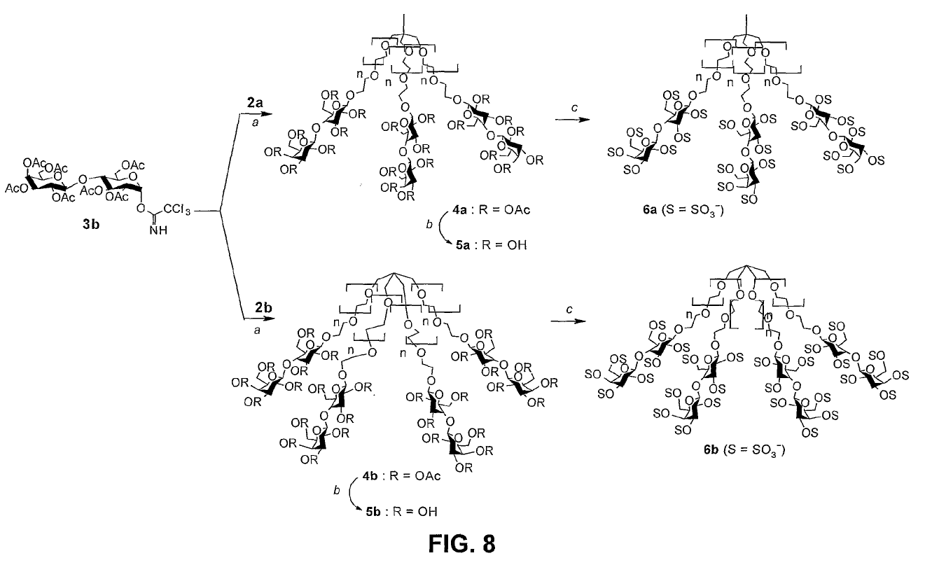

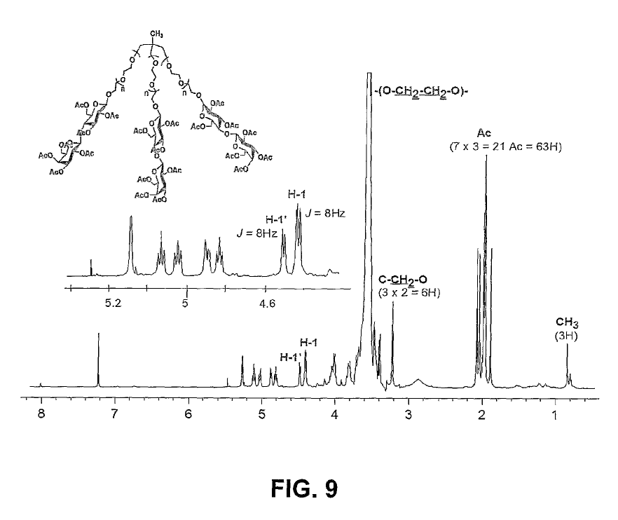

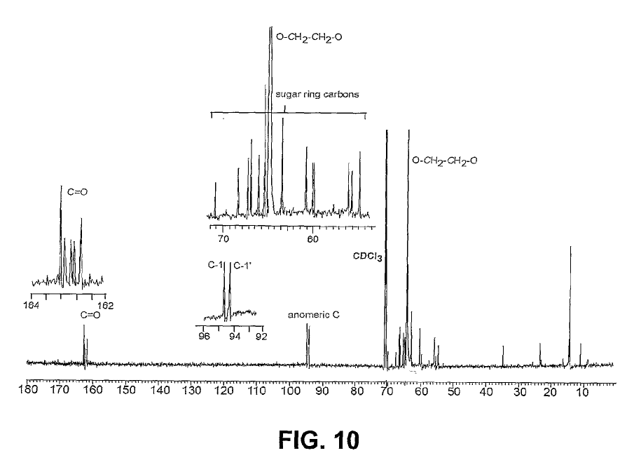

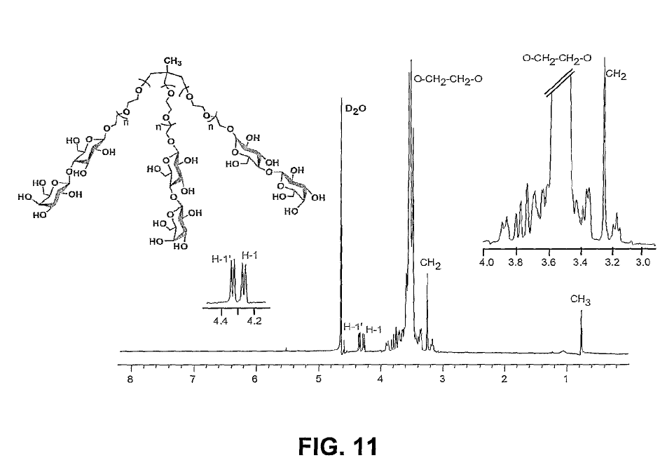

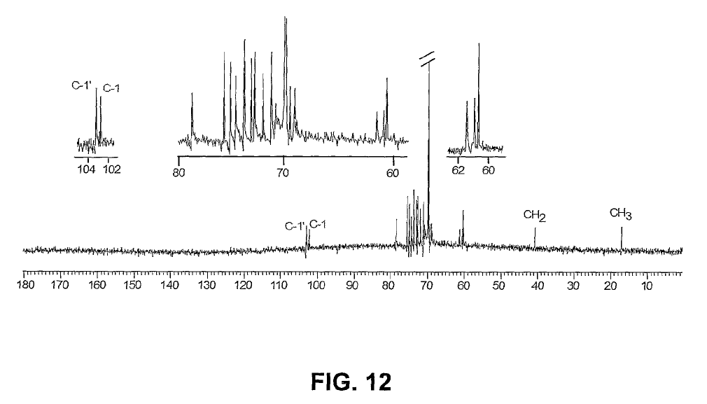

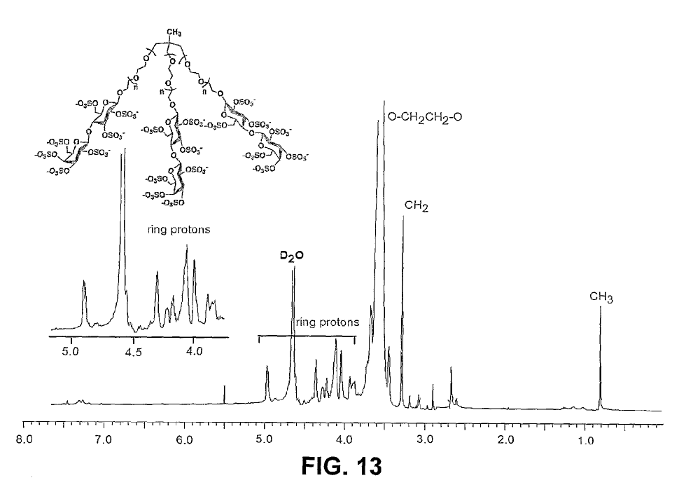

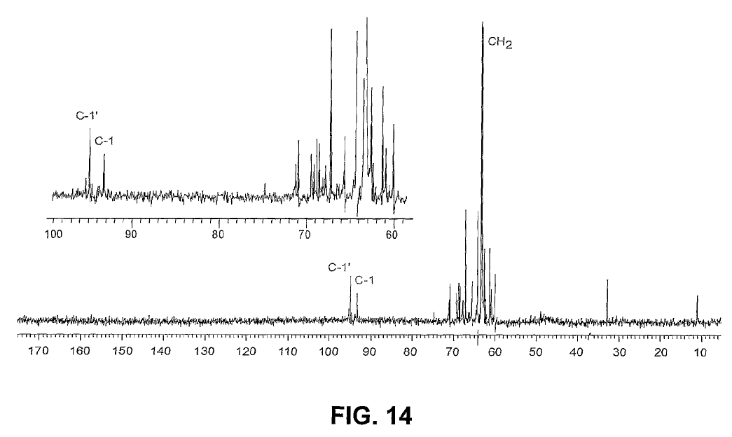

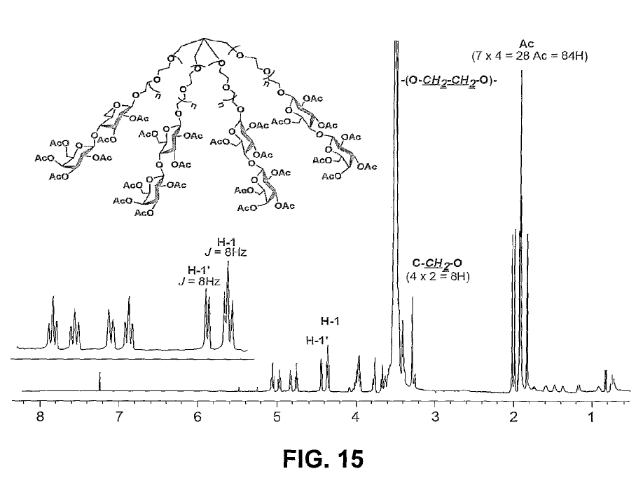

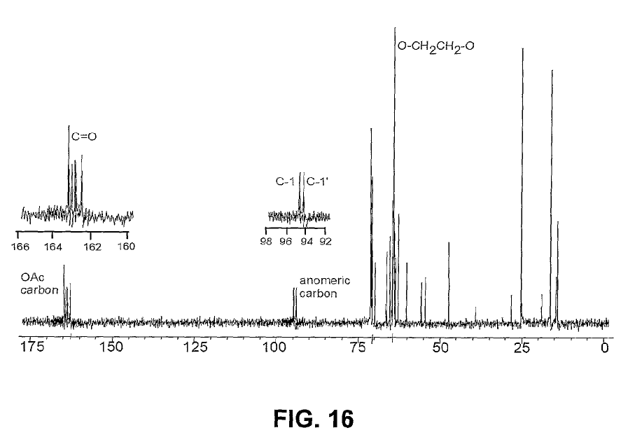

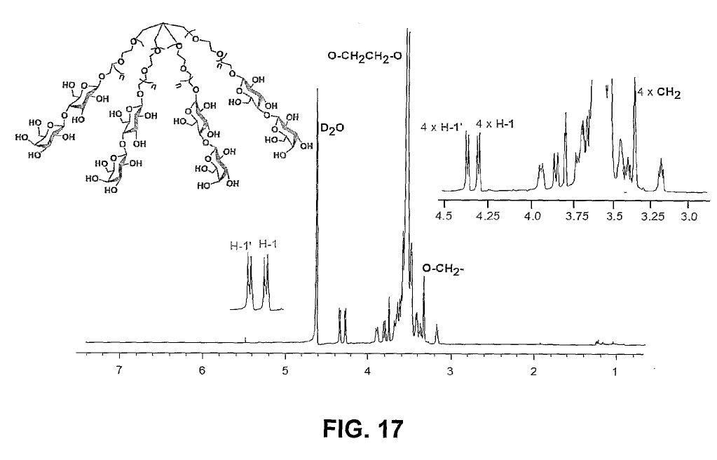

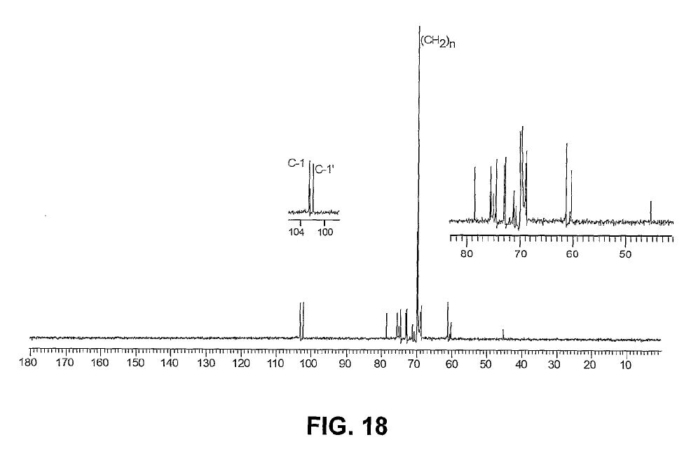

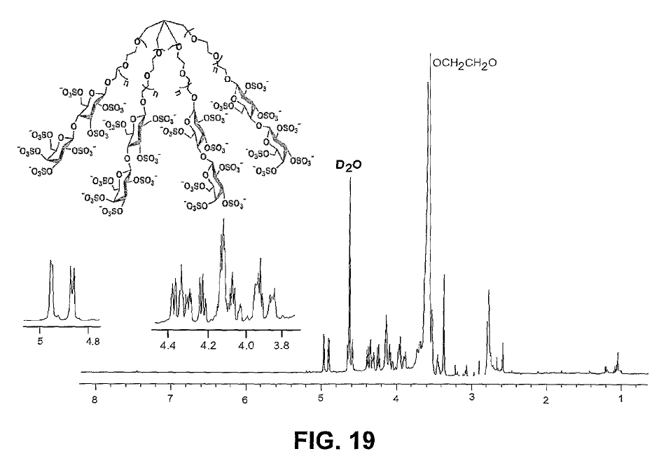

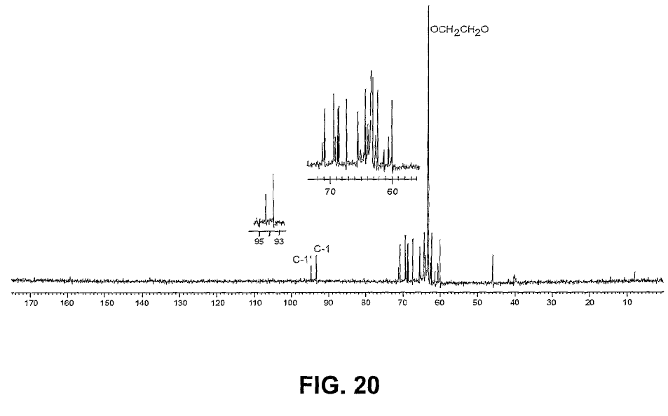

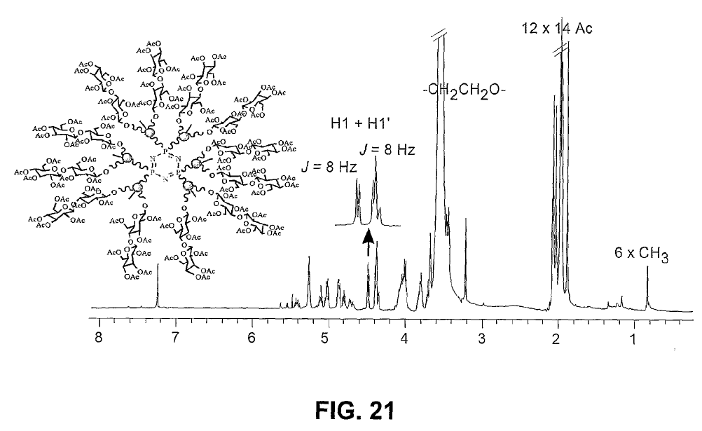

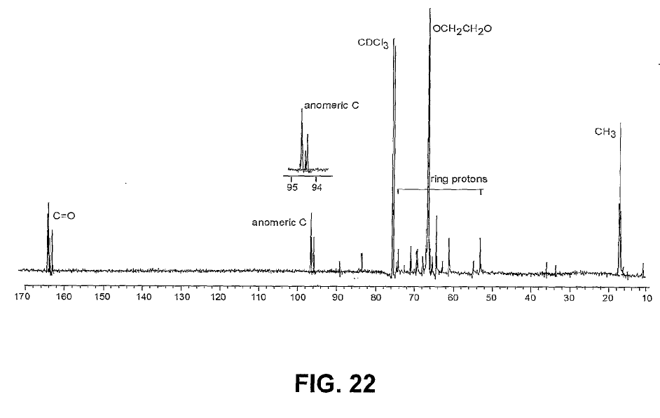

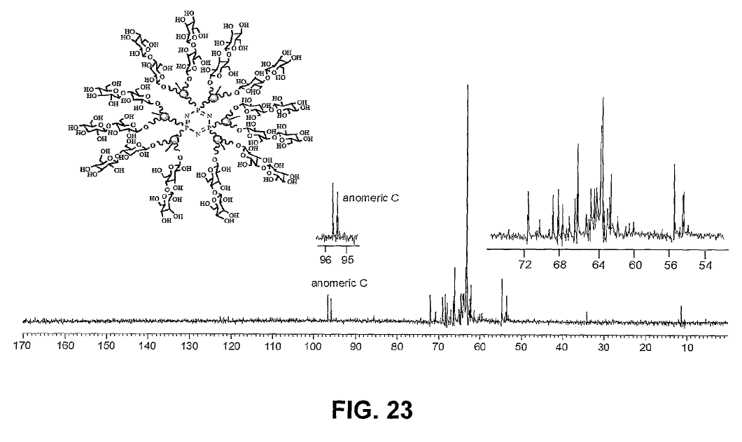

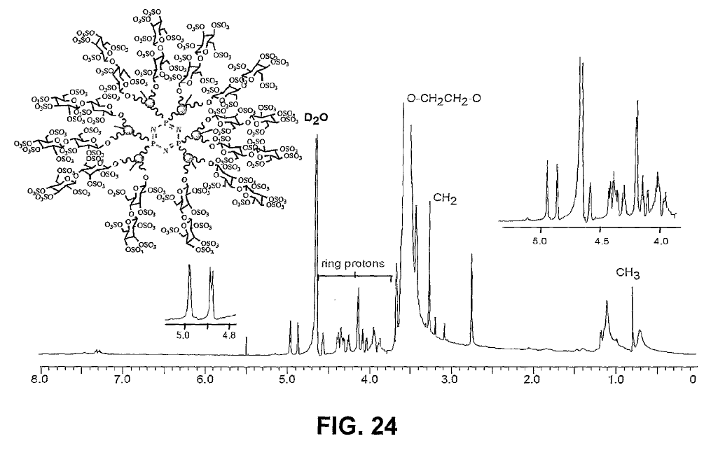

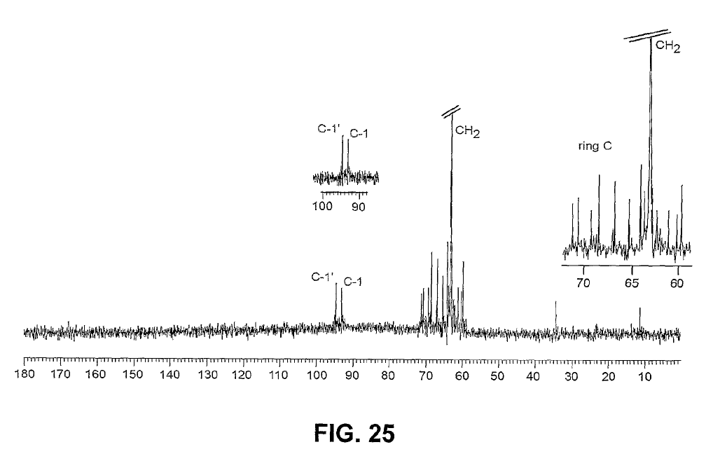

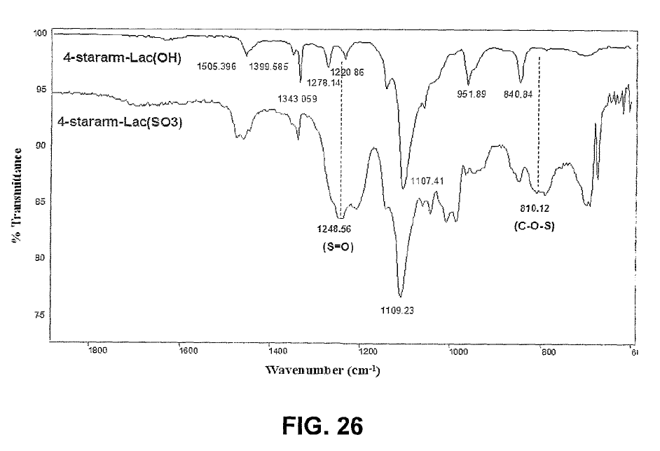

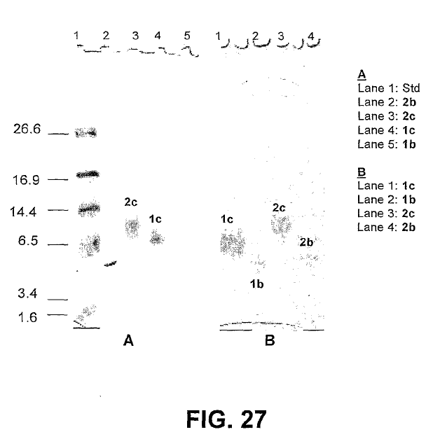

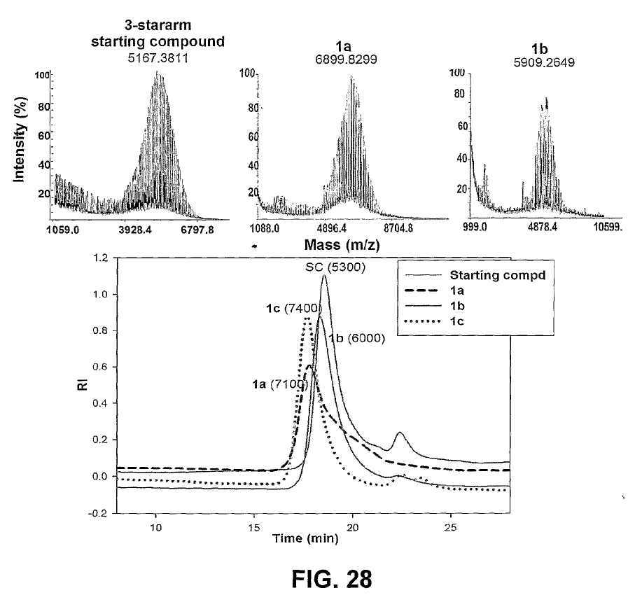

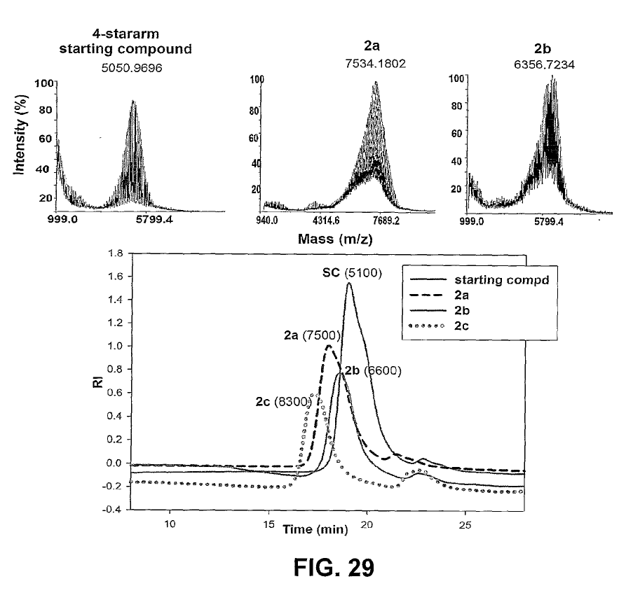

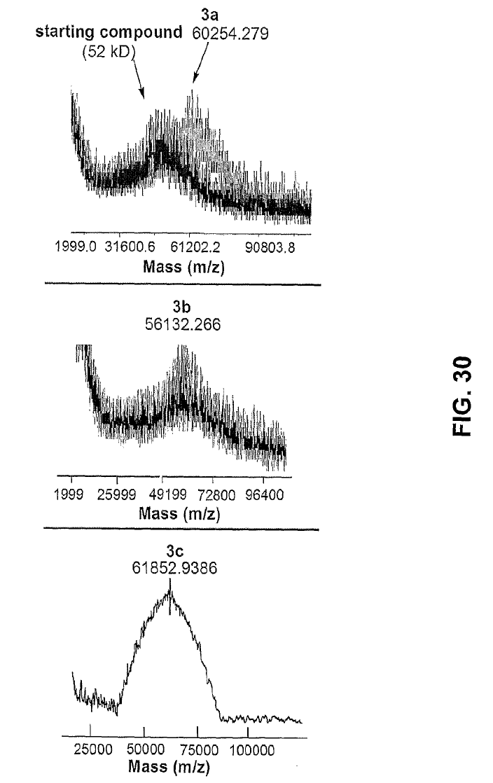

Suppression of experimental abdominal aortic aneurysms (AAAs) in mice by treatment with a polyethylene oxide glycopolymer antagonist of L-selectin was evaluated. The twelve arm multivalent heparinoid compound, SR-12 (see FIG. 1C) suppresses experimental AAA in mice. A statistically significant reduction in aortic diameter was observed in the group treated with compound SR-12. See FIG. 6 and the table below.

TABLE 1
Aortic Diameter Measurements.

| | | AD Pre | AD Post | AD Final |
|---|---|---|---|---|
| Treatment | n | (mm) | (mm) | (mm) |
| Control | 20 | 0.49 +/− 0.01 | 0.87 +/− 0.01 | 1.28 +/− 0.03 |
| | | (0.50) | (0.85) | (1.25) |
| Compound | 20 | 0.50 +/− 0.00 | 0.88 +/− 0.01 | 1.14 +/− 0.02 |
| | | (0.50) | (0.90) | (1.15) |
| Mann-Whitney | | NS | NS | P = 0.0015 |

### Methods.

In this example, an elastase infusion model was used in mice (strain C57BL/6, n=20). The mice were subjected to aortic elastase perfusion to induce experimental aortic aneurysms (Thompson and Baxter, 1999). Mice received 0.5 mg of compound SR-12 (also referred to as 3c) or 0.2 ml saline IV each day for the first 5 days after elastase infusion. Mice were sacrificed at 14 days. Aortic diameters (AD) prior to and immediately after elastase infusion, as well as at 14 days are reported.

### Further Studies.

The effectiveness of glycodendrimer SR-12 in limiting AAA formation and growth is determined. The frequency, size, and growth rate of aortic aneurysms is determined in both elastase infusion and angiotensin II (Ag II) murine models of AAA formation. Immunohistochemical studies are performed to characterize cellular and structural changes and MMP-2 and MMP-9 expression is examined by RT-PCR and gel zymography.

The capacity of SR-12 to limit chemokine binding to surface bound heparan sulfate and abrogate chemokine specific cell activation is defined. The ability of SR-12 to bind RANTES, MIP-1alpha, MCP-1, and SDF-1 and limit their interaction with surface bound heparan sulfate is determined by a surface plasmon resonance binding assay. Both kinetic rate constants and equilibrium constants can be characterized. Additionally, the ability of SR-12 to abrogate chemokine mediated cell activation is studied.

The ability is assessed of a carrier compound, sodium N-[8-(2-hydroxybenzoyl)amino]caprylate (SNAC), to facilitate effective biodistribution of SR-12 when both are administered orally. Effective plasma levels and the circulatory half-life of SR-12 are measured in mice after oral delivery either alone or in association with a carrier compound. Measured plasma levels and half-lives are compared to those achieved by intravenous delivery of the glycopolymer.

### STATEMENTS REGARDING INCORPORATION BY REFERENCE AND VARIATIONS

All references throughout this application, for example patent documents including issued or granted patents or equivalents; patent application publications; unpublished patent applications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Separate embodiments of the invention are also intended to be encompassed wherein the terms “comprising” or “comprise(s)” or “comprised” are optionally replaced with the terms, analogous in grammar, e.g.; “consisting/consist(s)” or “consisting essentially of/consist(s) essentially of” to thereby describe further embodiments that are not necessarily coextensive.

The invention has been described with reference to various specific and preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be applied to the practice of the invention as broadly disclosed herein without resort to undue experimentation. All art-known functional equivalents of compositions, methods, devices, device elements, materials, procedures and techniques described herein are intended to be encompassed by this invention. Whenever a range is disclosed, all subranges and individual values are intended to be encompassed as if separately set forth. This invention is not to be limited by the embodiments disclosed, including any shown in the drawings or exemplified in the specification, which are given by way of example or illustration and not of limitation. The scope of the invention shall be limited only by the claims.

### REFERENCES

Rele S M et al., J Am Chem Soc. 2005 Jul. 27; 127(29):10132-3. Dendrimer-like PEO glycopolymers exhibit anti-inflammatory properties.
Feng X—S et al., J Am Chem Soc. 2005; 127:10956-10966. Toward an easy access to dendrimer-like Poly(ethylene oxide)s.
Rele S M, Iyer S S, Baskaran S, Chaikof E L; J Org Chem. 2004 Dec. 24; 69(26):9159-70. Design and synthesis of dimeric heparinoid mimetics.
Gillies E R, Dy E, Frechet J M, Szoka F C. Biological evaluation of polyester dendrimer: poly(ethylene oxide) “bow-tie” hybrids with tunable molecular weight and architecture. Mol Pharm. 2005 March-April; 2(2):129-38.
U.S. Pat. Nos. 6,961,610; 6,902,812; 6,616,982; 5,275,838; 6,252,014; 6,184,313; 5,830,986; 5,648,186; 5,171,264; 6,479,001; 6,110,849; 6,046,305; 5,998,588; 5,462,990; 5,380,536; 6,117,453; 6,670,199; 6,113,906; 5,643,575.
20020123609; 20030181613; 20060002903; 20020182633; 20050164982; 20040180852; 20050238684; 20050164982; 20040180852.
PCT and foreign Patent Publications: WO0226867A2; WO0072052A1; EP0684962B1; WO05107828A2; WO00226867A2.
Sun X L, Grande D, Baskaran S, Hanson S R, Chaikof E L. Biomacromolecules. 2002 September-October; 3(5):1065-70.
(1) (a) Ulbrich, H.; Eriksson, E.; Lindbom, L. Trends Pharmacol. Sci. 2003, 24, 640-647.
(b) Ley, K. Trends Mol. Med. 2003, 9, 263-268.
(c) Hopfner, M.; Alban, S.; Schumacher, G.; Rothe, U.; Bendas, G. J. Pharm. Phamacol. 2003, 55, 697-706.
(d) Lowe, J. B.; Ward, P. A. J. Clin. InVest. 1997, 99, 822-826.
(2) (a) Sanders, W. J.; Gordon, E. J.; Oren, D.; Beck, P. J.; Ronen, A.; Kiessling, L. L. J. Biol. Chem. 1999, 274, 5271-5278.
(b) Reuter, J. et al. Bioconjugate Chem. 1999, 10, 271-278.
(c) Lin, C.-C.; Kimura, T.; Wu, S.-H.; Weitz-Schmidt, G.; Wong, C.-H. Biorg. Med. Chem. Lett. 1996, 6, 2755-2760.
(d) Spevak, W.; Foxall, C.; Charych, D.; Dasgupta, F.; Nagy, J. O. J. Med. Chem. 1996, 39, 1018-1020.
(e) DeFrees, S. A.; Phillips, L.; Guo, L.; Zalipsky, S. J. Am. Chem. Soc. 1996, 118, 6101-6104.
(f) Manning, D. D.; Strong, L.; Hu, X.; Beck, P.; Kiessling, L. L. Tetrahedron 1997, 53, 11937-11952.
(g) Thoma, G.; Duthaler, R. O.; Magnani, J. L.; Patton, J. T. J. Am. Chem. Soc. 2001, 123, 10113-10114. (h) Barbaro, J. Curr. Org. Chem. 2004, 8, 883-902.
(3) (a) Lundquist, J. J.; Toone, E. J. Chem. ReV. 2002, 102, 555-578.
(b) Mammen, M.; Choi, S.-K.; Whitesides, G. M. Angew, Chem., Int. Ed. 1998, 37, 2754-2794.
(4) (a) Aoyama, Y. Chem. sEur. J. 2004, 10, 588-593.
(b) Casnati, A.; Sansone, F.; Ungaro, R. Acc. Chem. Res. 2003, 36, 246-254.
(c) Shaunak, S. et al. Nat. Biotechnol. 2004, 22, 977-984.
(5) (a) Frey, H.; Haag, R. ReV. Mol. Biotechnol. 2002, 90, 257-267.
(b) Zalipsky, S. AdV. Drug Deliery ReV. 1995, 16, 157-182.
(6) (a) Hou, S.; Chaikof, E.; Taton, D.; Gnanou, Y. Macromolecules 2003, 36, 3874-3881.
(b) Angot, S.; Taton, D.; Gnanou, Y. Macromolecules 2000, 33, 5418-5424.
(c) Hou, S.; Taton, D.; Saule, M.; Logan, J.; Chaikof, E.; Gnanou, Y. Polymer 2003, 44, 5067-5074.
(7) Wang, L.; Brown, J.; Varki, A.; Esko, J. J. Clin. InVest. 2002, 110, 127-136.
(8) (a) Bruehl, R.; Bertozzi, C.; Rosen, S. J. Biol. Chem. 2000, 275, 32642-32648.
(b) Rosen, D. Am. J. Pathol. 1999, 155, 1013-1020.
(c) Galustian, C.; Lubineau, A.; le Narvor, C.; Kiso, M.; Brown, G.; Feizi, T. J. Biol. Chem. 1999, 274, 18213-18217.
(9) (a) Roy, R.; Park, W. C.; Zanini, D.; Foxall, C.; Srivastava, O. P. Carbohydr. Lett. 1997, 2, 259.
(b) Mowery, P.; Yang, Z.-Q.; Gordon, E.; Beck, D.; Spencer, A.; Alon, R.; Kiessling, L. Chem. Biol. 2004, 11, 725-732.
(10) Xie, X.; Rivier, A.-S.; Zakrzewicz, A.; Bernimoulin, M.; Zeng, X.-L.; Wessel, H. P.; Schapira, M.; Spertini, O. J. Biol. Chem. 2000, 275, 34818-34825.
Gilbert R. Upchurch et al. L-selectin-mediated neutrophil recruitment in experimental rodent aneurysm formation. Circulation, 2005, 112, 241-247.
Thompson R W, Baxter B T, 1999. Ann NY Acad Sci 878:159-78.

1. A branched glycopolymer comprising the formula: L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

2. The branched glycopolymer of claim 1 wherein L is a sulfated lactose.

3. The branched glycopolymer of any of claims 1-2 wherein A comprises polyethylene oxide.

4. The branched glycopolymer of any of claims 1-3 wherein X is the core selected from the group consisting of alkyl and a phosphazene group.

5. The branched glycopolymer of claim 4 wherein X is alkyl.

6. The branched glycopolymer of claim 4 wherein X is neopentyl.

7. The branched glycopolymer of claim 4 wherein X is a phosphazene group.

8. The branched glycopolymer of claim 7 wherein the phosphazene group is a cyclolinear phosphazene.

9. The branched glycopolymer of claim 8 wherein the cyclolinear phosphazene is a cyclic trimer.

10. The branched glycopolymer of any of claims 1-3 wherein there is a single branch generation.

11. The branched glycopolymer of any of claims 1-3 wherein there is a plurality of branch generations.

12. The branched glycopolymer of claim 11 wherein there are two branch generations.

13. The branched glycopolymer of claim 12 wherein there is a first branch generation of six arms and a second branch generation of two arms from each of the first generation, and wherein the total number of arms is 12.

14. The branched glycopolymer of claim 5 wherein n=3 or 4.

15. The branched glycopolymer of claim 7 wherein n=12.

16. The branched glycopolymer of claim 4 wherein n is from about 6 to about 96.

17. The branched glycopolymer of claim 4 wherein n is from about 12 to about 48.

18. The branched glycopolymer of claim 4 wherein m=from about 2 to about 600.

19. The branched glycopolymer of any of claims 1-3 wherein a value for n is selected from the group consisting of n=2, 3, 4, and 12.

20. A method of preparing a branched glycopolymer composition of a structural formula represented by L-[Aₘ]ₙ-X; wherein L is a saccharide or a saccharide derivative; A is a polymeric arm comprising a poly(alkylene oxide) where m is a number of monomeric units; n is a number of branching arms where n=2 to about 100; and X is a polymer core.

21. The method of claim 20 comprising the steps of providing an imidated lactose donor group and performing a Schmidt glycosidation coupling.

22. The method of claim 20 comprising anionic polymerization using a core-first approach.

23. The method of claim 20 comprising synthesis of a first generation of PEO arms on a phosphazene core using an arm-first approach.

24. The method of claim 23 further comprising synthesis of a second generation of PEO arms, wherein the second generation is directly polymerized onto the first generation.

25. The method of claim 23 further comprising synthesis of multiple further generations of PEO arms, wherein each further generation is directly polymerized onto the previous generation.

26. The method of claim 25 wherein a total number of generations is from about 3 to about 8.

27. A method of modifying an inflammatory condition comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

28. A method of modifying a cell adhesion event comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

29. A method of modifying a selectin-mediated interaction comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

30. The method of claim 29 wherein the selectin-mediated interaction involves a selectin selected from the group consisting of L-selectin, P-selectin, and E-selectin.

31. The method of claim 29 wherein the selectin-mediated interaction involves a selectin selected from the group consisting of L-selectin.

32. The method of claim 29 wherein L is the lactose derivative which is a sulfated lactose.

33. The method of claim 28 or 29 wherein said modifying occurs in vivo, in vitro, or ex vivo.

34. The method of claim 28 or 29 wherein said modifying occurs in vitro.

35. The method of claim 28 or 29 wherein said modifying occurs in vitro under hemodynamic flow conditions.

36. A method of therapy for abdominal aortic aneurysm comprising administering to a patient in need a compound of the formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core.

37. A medical device treated with a compound of formula L-[Aₘ]ₙ-X; wherein L is lactose or a lactose derivative; A is a polymeric arm comprising a poly(alkylene oxide) where m is a number of monomeric units; n is a number of branching arms where n=2 to about 100; and X is a polymer core; wherein said treated device has at least a portion of a surface that is capable of contacting a patient.

38. The medical device of claim 36 selected from the group consisting of a stent, embolization coil, vascular graft, or other biomedical device capable of exposure to a patient.

39. A medical device, cell, tissue, or organ further comprising a film, gel, or other coating with the compound of any of claims 1-19.

40. A branched glycopolymer comprising the formula: L-[A2ₘ₂]ₙ₂-[A1ₘ₁]ₙ₁-X; wherein L is lactose or a lactose derivative; A1 and A2 are polymeric arms comprising a poly(alkylene oxide) where m1 and m2 are numbers of monomeric units; n1 and n2 are numbers of branching arms where n1=2 to about 100 and n2=2 to about 100; and X is a polymer core.

41. The branched glycopolymer of claim 40 wherein m1 is from about 2 to about 400 and m2 is from about 2 to about 100.

42. The branched glycopolymer of claim 40 wherein m1 is from about 100 to about 150 and m2 is from about 10 to about 25.

43. The branched glycopolymer of claim 40 wherein A1 and A2 are each poly(ethylene oxide); m1 is from about 100 to about 150; n1 is 6; m2 is from about 10 to about 30; n2 is 2, and L is selected from the group consisting of lactose, sulfated lactose, and other derivatized lactose.

44. A branched glycopolymer comprising the formula: S-[Aₘ₂]ₙ₂-[Aₘ₁]ₙ₁-X; wherein S is a saccharide or a saccharide derivative; A is a polymeric arm comprising a poly(alkylene oxide) where m1 and m2 are numbers of monomeric units; n1 and n2 are numbers of branching arms where n1=2 to about 100 and n2=2 to about 100; and X is a polymer core.

45. The branched glycopolymer of claim 1 wherein m is from about 20 to about 50.

46. The branched glycopolymer of claim 1 wherein m is from about 30 to about 45.

47. The branched glycopolymer of claim 1 wherein m is from about 20 to about 50 and n is 3 or 4.

48. The invention substantially as herein described and illustrated.

49. A new compound, substantially as herein described.

50. A new use of a compound, substantially as herein described.

51. A substance or composition for a new use in a method of treatment, substantially as herein described.

52. A new process for preparing a compound, substantially as herein described.
